(19) United States
(12) Patent Application Publication
(10) Pub. No.: US 2026/0027019 A1
(43) Pub. Date: Jan. 29, 2026

(54) TRANSPARENT COSMETIC COMPOSITION CONTAINING WATER PHASE AND OIL PHASE

(71) Applicant: AMOREPACIFIC CORPORATION, Seoul (KR)

(72) Inventors: Seung Hwan YI, Yongin-si (KR); Kyung Nam KIM, Yongin-si (KR)

(73) Assignee: AMOREPACIFIC CORPORATION, Seoul (KR)

(21) Appl. No.: 19/271,957

(22) Filed: Jul. 17, 2025

(30) Foreign Application Priority Data
Jul. 23, 2024 (KR) 10-2024-0097434

(51) Int. Cl. A61K 8/06; A61Q 17/04

(52) U.S. Cl. CPC A61K 8/06; A61Q 17/04; A61K 2800/262; A61K 2800/30; A61K 2800/805; A61K 2800/85

(57) ABSTRACT

A cosmetic composition including a water phase part and an oil phase part, wherein the water phase part and the oil phase part are mixed, and the cosmetic composition is a transparent formulation. Specifically, the cosmetic composition of the present disclosure is prepared in a transparent formulation in which the water phase and the oil phase form a bicontinuous phase without an emulsifier, and does not cause white cast due to a UV blocking agent when applied to the skin.

### CROSS REFERENCE TO RELATED APPLICATION

The present application claims priority to Korean Patent Application No. 10-2024-0097434, filed Jul. 23, 2024, the entire contents of which are hereby incorporated by this reference.

### BACKGROUND OF THE INVENTION

### Field of the Invention

One aspect of the present disclosure relates to a transparent cosmetic composition containing the water phase and the oil phase.

### Description of the Related Art

Ultraviolet rays emitted from sunlight act as a main cause of erythema, edema, freckles, or skin cancer on the skin, and accordingly, many studies on various skin diseases caused by ultraviolet rays have been actively conducted recently.

In general, ultraviolet rays are divided into UV-A, UV-B and UV-C according to their wavelengths, with wavelengths of 320 nm to 400 nm being classified as UV-A, wavelengths of 280 nm to 320 nm being classified as UV-B, and wavelengths of 200 nm and 280 nm being classified as UV-C. UV-A is known to penetrate into the dermis of the skin, cause skin cancer, and promote wrinkles and melanin formation, thereby inducing skin aging and skin irritation, while UV-B is known to penetrate into the epidermis of the skin, thereby causing erythema, freckles, or edema. UV-C disappears without reaching the ground, while passing through the ozone layer.

In order to prevent or ameliorate the above-described skin diseases associated with sun exposure, consumers are increasingly interested in UV blocking products.

Meanwhile, recently in the market for UV blocking cosmetics, there has been an increasing demand from customers for cosmetics that are simultaneously satisfy naturalness and skin texture care, and there is a growing need for transparent UV blocking cosmetics that can be used by anyone without white cast and satisfy people of various ethnicities.

### SUMMARY OF THE INVENTION

While providing a cosmetic composition having a UV blocking function and developing a composition capable of providing a transparent skin expression without white cast, the present inventors have prepared a transparent cosmetic composition in which an oil phase and a water phase are mixed by adjusting the viscosities of the water and oil phases.

Therefore, in one aspect, an object of the present disclosure is to provide a cosmetic composition including a water phase part and an oil phase part, wherein the water phase part and the oil phase part are mixed, and the cosmetic composition is a transparent formulation.

In one aspect, the present disclosure provides a cosmetic composition including a water phase part and an oil phase part, wherein the water phase part and the oil phase part are mixed, and the cosmetic composition is a transparent formulation.

In an exemplary embodiment, the composition may be for blocking ultraviolet rays.

In an exemplary embodiment, the viscosity of the composition may be 2000 cps to 15000 cps (Brookfield viscometer) at 30° C.

In an exemplary embodiment, the composition may allow the water phase part and the oil phase part to be mixed to form a bicontinuous phase.

In an exemplary embodiment, the water phase part may include a water-dispersible organic UV blocking agent and a powder, and the oil phase part may include an oil-dispersible organic UV blocking agent and a powder.

In an exemplary embodiment, the powder may be silica or cellulose.

In an exemplary embodiment, the weight of the powder included in the water phase part may be 0.5 wt % to 3.0 wt % relative to the entire composition, and the weight of the powder included in the oil phase part may be 0.5 wt % to 3.0 wt % relative to the entire composition.

In an exemplary embodiment, the water-dispersible organic UV blocking agent may include one or more selected from the group consisting of phenylbenzimidazole sulfonic acid and terephthalylidene dicamphor sulfonic acid.

In an exemplary embodiment, the water-dispersible organic UV blocking agent may be included in an amount of 1 wt % to 13 wt % relative to the total weight of the composition.

In an exemplary embodiment, the oil-dispersible organic UV blocking agent may include one or more selected from the group consisting of octocrylene, ethylhexyl salicylate, homosalate, bis-ethylhexyloxyphenol methoxyphenyl triazine, polysilicone-15, ethylhexyl triazone, butyl methoxydibenzoylmethane, drometrizole trisiloxane, and diethylamino hydroxybenzoyl hexyl benzoate.

In an exemplary embodiment, the oil-dispersible organic UV blocking agent may be included in an amount of 5 wt % to 35 wt % relative to the total weight of the composition.

In an exemplary embodiment, the mixing weight ratio of the oil phase part to the water phase part may be 1:1 to 10:1.

In an exemplary embodiment, the water phase part may be thickened by further including a water phase gelling agent.

In an exemplary embodiment, the water phase gelling agent may include one or more selected from gums and mannan.

In an exemplary embodiment, the water phase gelling agent may include one or more selected from the group consisting of a carbomer, sodium polyacryloyldimethyl taurate, polyacrylate crosspolymer-6, xanthan gum, guar gum, locust bean gum, gellan gum, galactomannan, glucomannan, agar, carrageenan, a Sphingomonas ferment extract, acrylates/C10-30 alkyl acrylate crosspolymer, and an acrylates crosspolymer.

In an exemplary embodiment, the water phase part may further include a pH adjuster or neutralizer.

In an exemplary embodiment, the pH adjuster or neutralizer may include one or more selected from the group consisting of triethanolamine, aminomethyl propanol, ethanolamine, ammonium hydroxide, and tromethamine.

In an exemplary embodiment, the oil phase part may further include an elastomer.

In an exemplary embodiment, the elastomer may be a silicone gel.

In an exemplary embodiment, the silicone gel may include one or more selected from the group consisting of dimethicone/dimethicone crosspolymer, isododecane/dimethicone/bis-isobutyl PPG-20 crosspolymer, and dimethicone/vinyl dimethicone crosspolymer.

In an exemplary embodiment, the elastomer may be included in an amount of 5 wt % to 35 wt % relative to the total weight of the composition.

In one aspect, the composition of the present disclosure has an excellent effect of suppressing the white cast.

In one aspect, the composition of the present disclosure can provide a natural, transparent skin expression.

In one aspect, the composition of the present disclosure has an excellent effect of blocking ultraviolet rays.

In one aspect, the composition of the present disclosure includes both an oil phase and a water phase and is simultaneously embodied as a transparent formulation.

In one aspect, the composition of the present disclosure can maintain a stable formulation even without including an emulsifier, and thus, has excellent water resistance.

### BRIEF DESCRIPTION OF THE DRAWINGS

FIG. 1 is a schematic view of an emulsion formulation in the related art and a transparent composition formulation according to an exemplary embodiment of the present disclosure.

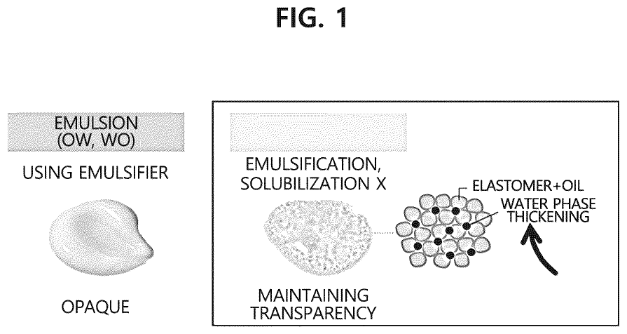

FIG. 2A shows the results of a comparative experiment on the appearance transparency of formulations of Example 1 and Comparative Example 1, according to whether or not a surfactant is included.

FIG. 2B shows the results of a comparative experiment on the appearance transparency of formulations of Example 2 and Comparative Example 2, according to whether or not a surfactant is included.

FIG. 3 shows the results of a comparative experiment on transparency during the application of Example 1 and Comparative Example 1.

FIG. 4 shows the results of a comparative experiment on water resistance for Example 1 and Comparative Example 1.

### DETAILED DESCRIPTION OF THE INVENTION

With respect to the terms used in the present specification, general terms currently and widely used are selected in consideration of function in the present disclosure, but the terms may vary according to an intention of a technician skilled in the art, a precedent, or the advent of a new technology, and the like. Further, in a specific case, there is also a term arbitrarily chosen by the applicant, and in this case, the meanings thereof will be described in detail in the corresponding part of the Detailed Description of the present disclosure. Accordingly, the term used in the present specification should not be defined merely as a simple name of the term, but should be defined based on the meaning of the term and overall content of the present disclosure.

Unless defined otherwise, all terms used herein, including technical or scientific terms, have the same meaning as commonly understood by a person with ordinary skill in the art to which the present disclosure pertains. Generally understood terms should be interpreted as having the same meaning as they have in the context of the related art, and should not be interpreted in an ideal or excessively formal meaning unless expressly defined in the present disclosure.

Numerical ranges are inclusive of the numerical values defined in the present disclosure. Every maximum numerical limitation given throughout the present specification includes all lower numerical limitations as if the lower numerical limitation were expressly written. Every minimum numerical limitation given throughout the present specification includes all higher numerical limitations as if the higher numerical limitation were expressly written. Any numerical limitation given throughout the present specification shall include all the better numerical ranges within the broader numerical range, as if the narrower numerical limitation were expressly written.

As used herein, the words “comprising,” “having,” and “containing,” are inclusive or open-ended and do not exclude additional unrecited elements or method steps. As used herein, the term “or combinations thereof” refers to all permutations and combinations of items listed prior to the term. For example, “A, B, C, or combinations thereof” means A, B, C, AB, AC, BC or ABC, and it is intended to include at least one of BA, CA, CB, CBA, BCA, ACB, BAC or CAB where order is important in a particular context. With this example, combinations containing repetitions of one or more items or terms may be included, such as BB, AAA, MB, BBC, AAABCCCC, CBBAAA, CABABB, and the like. A person of ordinary skill in the art will understand that there is typically no limit to the number of items or terms in any combination, unless the context makes it clear otherwise.

Hereinafter, exemplary embodiments of the present disclosure will be described in detail.

In one aspect, the present disclosure may provide a cosmetic composition including a water phase part and an oil phase part, wherein the water phase part and the oil phase part are mixed, and the cosmetic composition is a transparent formulation.

The term “water phase part” refers to a part that includes a water phase solvent and a plurality of particles dissolved on the water phase. For example, the water phase solvent may include ionized water, but is not limited thereto.

The term “oil phase part” refers to a part that includes an oil phase solvent and a plurality of particles dissolved on the oil phase. For example, the oil phase solvent may include dibutyl adipate, butyloctyl salicylate, or a mixture thereof, but is not limited thereto.

The term “transparent formulation” is a concept including all formulations that are not opaque, and includes, for example, all formulations that have a transparent or translucent appearance.

The appearance of the cosmetic composition may contain a cosmetically or dermatologically acceptable medium or base. It is any formulation suitable for topical application, and may be provided in the form of, for example, a solution, a gel, a solid, a kneaded anhydrous product, an emulsion obtained by dispersing an oil phase in the form of a water phase, a suspension, a microemulsion, a microcapsule, microgranules or an ionic (liposome) and a non-ionic vesicular dispersing agent, or in the form of a cream, a skin toner, a lotion, a powder, an ointment, a spray, or a conceal stick. These compositions may be prepared by a typical method in the field.

The cosmetic composition is not particularly limited in its formulation, and may be formulated into a cosmetic such as, for example, a softening lotion, an astringent lotion, a nourishing lotion, a nourishing cream, a massage cream, an essence, an eye cream, an eye essence, a cleansing cream, a cleansing foam, a cleansing water, a pack, a powder, a body lotion, a body cream, a body oil, or a body essence.

When the formulation of the cosmetic composition is a paste, a cream or a gel, an animal oil, a vegetable oil, a wax, paraffin, starch, traganth, a cellulose derivative, a polyethylene glycol, silicone, bentonite, silica, talc, zinc oxide, or the like may be used as a carrier ingredient.

When the formulation of the cosmetic composition is a powder or a spray, lactose, talc, silica, aluminum hydroxide, calcium silicate, or a polyamide powder may be used as the carrier ingredient, and in particular, when the formulation of the present disclosure is a spray, the formulation may additionally include a propellant such as a chlorofluorohydrocarbon, propane/butane or dimethyl ether.

The cosmetic composition according to an exemplary embodiment may further include functional additives and ingredients included in general cosmetic compositions. The functional additive may include one or more ingredients selected from the group consisting of a water-soluble vitamin, an oil-soluble vitamin, a polymeric peptide, a polymeric polysaccharide, a sphingolipid, and a seaweed extract.

The cosmetic composition may be blended with ingredients included in general cosmetic compositions, if necessary, along with the functional additives. Examples of other blended ingredients which may be contained include oil and fat ingredients, a moisturizer, an emollient, organic and inorganic pigments, an organic powder, a preservative, a bactericide, an antioxidant, a plant extract, a pH adjuster, an alcohol, a colorant, a fragrance, a circulation accelerator, a cooling agent, an antiperspirant, purified water, and the like.

In an exemplary embodiment, the composition may be for blocking ultraviolet rays.

In the composition of the present disclosure, the viscosities of the water phase and the oil phase are adjusted to be chemically or physically similar, so that the water phase and the oil phase may be mixed uniformly without the addition of an emulsifier. The viscosity of each phase may be selected without any limitation by those skilled in the art according to the viscosity of a desired cosmetic material.

In an exemplary embodiment, the viscosity of the composition may be 2000 cps to 15000 cps (Brookfield viscometer) at 30° C. For example, the viscosity of the composition may be 2000 cps or more, 2500 cps or more, 3000 cps or more, 3500 cps or more, 4000 cps or more, 4500 cps or more, 5000 cps or more, 5500 cps or more, 6000 cps or more, 6500 cps or more, 7000 cps or more, 7500 cps or more, 8000 cps or more, 8500 cps or more, 9000 cps or more, 9500 cps or more, 10000 cps or more, 10500 cps or more, 11000 cps or more, 11500 cps or more, 12000 cps or more, 12500 cps or more, 13000 cps or more, 13500 cps or more, 14000 cps or more, 14500 cps or more, or 15000 cps or more, at 30° C., but is not limited thereto. In addition, the viscosity of the composition may be 15000 cps or less, 14500 cps or less, 14000 cps or less, 13500 cps or less, 13000 cps or less, 12500 cps or less, 12000 cps or less, 11500 cps or less, 11000 cps or less, 10500 cps or less, 10000 cps or less, 9500 cps or less, 9000 cps or less, 8500 cps or less, 8000 cps or less, 7500 cps or less, 7000 cps or less, 6500 cps or less, 6000 cps or less, 5500 cps or less, 5000 cps or less, 4500 cps or less, 4000 cps or less, 3500 cps or less, 3000 cps or less, 2500 cps or less, or 2000 cps or less, at 30° C., but is not limited thereto.

In an exemplary embodiment, the composition may allow the water phase part and the oil phase part to be mixed to form a bicontinuous phase. The term “bicontinuous phase” refers to a state in which neither the water phase part nor the oil phase part forms a continuous phase or dispersed phase, and the phases are mixed to form a stable layer.

In an exemplary embodiment, the water phase part may include a water-dispersible organic UV blocking agent and a powder, and the oil phase part may include an oil-dispersible organic UV blocking agent and a powder. The powder may include any powder without any limitation as long as the viscosity of the water phase or oil phase can be adjusted by those skilled in the art.

In an exemplary embodiment, the powder may be silica or cellulose.

In an exemplary embodiment, the weight of the powder included in the water phase part may be 0.5 wt % to 3.0 wt % relative to the entire composition, and the weight of the powder included in the oil phase part may be 0.5 wt % to 3.0 wt % relative to the entire composition.

In an exemplary embodiment, the water-dispersible organic UV blocking agent may include one or more selected from the group consisting of phenylbenzimidazole sulfonic acid and terephthalylidene dicamphor sulfonic acid.

In an exemplary embodiment, the water-dispersible organic UV blocking agent may be included in an amount of 1 wt % to 13 wt % relative to the total weight of the composition. For example, the water-dispersible organic UV blocking agent may be included in an amount of 1 wt % or more, 1.5 wt % or more, 2 wt % or more, 2.5 wt % or more, 3 wt % or more, 3.5 wt % or more, 4 wt % or more, 4.5 wt % or more, 5 wt % or more, 5.5 wt % or more, 6 wt % or more, 6.5 wt % or more, 7 wt % or more, 7.5 wt % or more, 8 wt % or more, 8.5 wt % or more, 9 wt % or more, 9.5 wt % or more, 10 wt % or more, 10.5 wt % or more, 11 wt % or more, 11.5 wt % or more, 12 wt % or more, or 12.5 wt % or more, relative to the total weight of the composition, but the amount is not limited thereto. Furthermore, the water-dispersible organic UV blocking agent may be included in an amount of 13 wt % or less, 12.5 wt % or less, 12 wt % or less, 11.5 wt % or less, 11 wt % or less, 10.5 wt % or less, 10 wt % or less, 9.5 wt % or less, 9 wt % or less, 8.5 wt % or less, 8 wt % or less, 7.5 wt % or less, 7 wt % or less, 6.5 wt % or less, 6 wt % or less, 5.5 wt % or less, 5 wt % or less, 4.5 wt % or less, 4 wt % or less, 3.5 wt % or less, 3 wt % or less, 2.5 wt % or less, 2 wt % or less, or 1.5 wt % or less, relative to the total weight of the composition, but the amount is not limited thereto.

In an exemplary embodiment, the oil-dispersible organic UV blocking agent may include one or more selected from the group consisting of octocrylene, ethylhexyl salicylate, homosalate, bis-ethylhexyloxyphenol methoxyphenyl triazine, polysilicone-15, ethylhexyl triazone, butyl methoxydibenzoylmethane, drometrizole trisiloxane, and diethylamino hydroxybenzoyl hexyl benzoate.

In an exemplary embodiment, the oil-dispersible organic UV blocking agent may be included in an amount of 5 wt % to 35 wt % relative to the total weight of the composition. For example, the oil-dispersible organic UV blocking agent may be included in an amount of 5 wt % or more, 6 wt % or more, 7 wt % or more, 8 wt % or more, 9 wt % or more, 10 wt % or more, 11 wt % or more, 12 wt % or more, 13 wt % or more, 14 wt % or more, 15 wt % or more, 16 wt % or more, 17 wt % or more, 18 wt % or more, 19 wt % or more, 20 wt % or more, 21 wt % or more, 22 wt % or more, 23 wt % or more, 24 wt % or more, 25 wt % or more, 26 wt % or more, 27 wt % or more, 28 wt % or more, 29 wt % or more, 30 wt % or more, 31 wt % or more, 32 wt % or more, 33 wt % or more, or 34 wt % or more, relative to the total weight of the composition, but the amount is not limited thereto. Further, for example, the oil-dispersible organic UV blocking agent may be included in an amount of 35 wt % or less, 34 wt % or less, 33 wt % or less, 32 wt % or less, 31 wt % or less, 30 wt % or less, 29 wt % or less, 28 wt % or less, 27 wt % or less, 26 wt % or less, 25 wt % or less, 24 wt % or less, 23 wt % or less, 22 wt % or less, 21 wt % or less, 20 wt % or less, 19 wt % or less, 18 wt % or less, 17 wt % or less, 16 wt % or less, 15 wt % or less, 14 wt % or less, 13 wt % or less, 12 wt % or less, 11 wt % or less, 10 wt % or less, 9 wt % or less, 8 wt % or less, 7 wt % or less, or 6 wt % or less, relative to the total weight of the composition, but the amount is not limited thereto.

In an exemplary embodiment, the mixing weight ratio of the oil phase part to the water phase part may be 1:1 to 10:1. For example, the mixing weight ratio of the oil phase part to the water phase part may be 1:1 or more, 1.1:1 or more, 1.2:1 or more, 1.3:1 or more, 1.4:1 or more, 1.5:1 or more, 1.6:1 or more, 1.7:1 or more, 1.8:1 or more, 1.9:1 or more, 2:1 or more, but is not limited thereto. In addition, the mixing weight ratio of the oil phase part to the water phase part may be 10:1 or less, 9.5:1 or less, 9:1 or less, 8.5:1 or less, 8:1 or less, 7.5:1 or less, 7:1 or less, 6.5:1 or less, 6:1 or less, 5.5:1 or less, 5:1 or less, 4.5:1 or less, 4:1 or less, 3.5:1 or less, 3:1 or less, 2.8:1 or less, 2.6:1 or less, 2.4:1 or less, or 2.2:1 or less, but is not limited thereto. Those skilled in the art may appropriately select the mixing weight ratio of the oil phase part to the water phase part in order to adjust the viscosity of the composition.

In an exemplary embodiment, the water phase part may be thickened by further including a water phase gelling agent. The water phase gelling agent may be included without any limitation as long as the water phase gelling agent can be dissolved in the water phase to increase the viscosity by those skilled in the art.

In an exemplary embodiment, the water phase gelling agent may include one or more selected from gums and mannan.

In an exemplary embodiment, the water phase gelling agent may include one or more selected from the group consisting of a carbomer, sodium polyacryloyldimethyl taurate, polyacrylate crosspolymer-6, xanthan gum, guar gum, locust bean gum, gellan gum, galactomannan, glucomannan, agar, carrageenan, a Sphingomonas ferment extract, acrylates/C10-30 alkyl acrylate crosspolymer, and an acrylates crosspolymer.

In an exemplary embodiment, the water phase part may further include a pH adjuster or neutralizer.

In an exemplary embodiment, the pH adjuster or neutralizer may include one or more selected from the group consisting of triethanolamine, aminomethyl propanol, ethanolamine, ammonium hydroxide, and tromethamine.

In an exemplary embodiment, the pH adjuster or neutralizer may be included in an amount of 80 wt % or more by weight relative to the weight of the water-dispersible organic UV blocking agent. For example, the pH adjuster or neutralizer may be included in an amount of 80 wt % or more, 80.5 wt % or more, 81 wt % or more, 81.5 wt % or more, 82 wt % or more, 82.5 wt % or more, 83 wt % or more, 83.5 wt % or more, 84 wt % or more, 84.5 wt % or more, 85 wt % or more, 85.5 wt % or more, 86 wt % or more, 86.5 wt % or more, 87 wt % or more, or 87.5 wt % or more, relative to the weight of the water-dispersible organic UV blocking agent, but the amount is not limited thereto.

In an exemplary embodiment, the oil phase part may further include an elastomer.

In an exemplary embodiment, the elastomer may be a silicone gel.

In an exemplary embodiment, the silicone gel may include one or more selected from the group consisting of dimethicone/dimethicone crosspolymer, isododecane/dimethicone/bis-isobutyl PPG-20 crosspolymer, and dimethicone/vinyl dimethicone crosspolymer.

In an exemplary embodiment, the elastomer may be included in an amount of 5 wt % to 35 wt % relative to the total weight of the composition. For example, the elastomer may be included in an amount of 5 wt % or more, 6 wt % or more, 7 wt % or more, 8 wt % or more, 9 wt % or more, 10 wt % or more, 11 wt % or more, 12 wt % or more, 13 wt % or more, 14 wt % or more, 15 wt % or more, 16 wt % or more, 17 wt % or more, 18 wt % or more, 19 wt % or more, 20 wt % or more, 21 wt % or more, 22 wt % or more, 23 wt % or more, 24 wt % or more, 25 wt % or more, 26 wt % or more, 27 wt % or more, 28 wt % or more, 29 wt % or more, 30 wt % or more, 31 wt % or more, 32 wt % or more, 33 wt % or more, or 34 wt % or more, relative to the total weight of the composition, but the amount is not limited thereto. Furthermore, for example, the elastomer may be included in an amount of 35 wt % or less, 34 wt % or less, 33 wt % or less, 32 wt % or less, 31 wt % or less, 30 wt % or less, 29 wt % or less, 28 wt % or less, 27 wt % or less, 26 wt % or less, 25 wt % or less, 24 wt % or less, 23 wt % or less, 22 wt % or less, 21 wt % or less, 20 wt % or less, 19 wt % or less, 18 wt % or less, 17 wt % or less, 16 wt % or less, 15 wt % or less, 14 wt % or less, 13 wt % or less, 12 wt % or less, 11 wt % or less, 10 wt % or less, 9 wt % or less, 8 wt % or less, 7 wt % or less, or 6 wt % or less, relative to the total weight of the composition, but the amount is not limited thereto.

In an exemplary embodiment, the composition may not include a surfactant or emulsifier.

The present disclosure may provide the following embodiments as an example.

A first embodiment may provide a cosmetic composition including a water phase part and an oil phase part, wherein the water phase part and the oil phase part are mixed, and the cosmetic composition is a transparent formulation.

A second embodiment may provide the composition of the first embodiment, wherein the composition is for blocking ultraviolet rays.

A third embodiment may provide the composition of the first embodiment, which a viscosity of the composition is 2000 cps to 15000 cps (Brookfield viscometer) at 30° C.

A fourth embodiment may provide the composition of the first embodiment, wherein the composition allows the water phase part and the oil phase part to be mixed to form a bicontinuous phase.

A fifth embodiment may provide the composition of the first embodiment, wherein the water phase part includes a water-dispersible organic UV blocking agent and a powder, and the oil phase part includes an oil-dispersible organic UV blocking agent and a powder.

A sixth embodiment may provide the composition of the fifth embodiment, wherein the powder is silica or cellulose.

A seventh embodiment may provide the composition of the fifth embodiment, wherein a weight of the powder included in the water phase part is 0.5 wt % to 3.0 wt % relative to the entire composition, and a weight of the powder included in the oil phase part is 0.5 wt % to 3.0 wt % relative to the entire composition.

An eighth embodiment may provide the composition of the fifth embodiment, wherein the water-dispersible organic UV blocking agent includes one or more selected from the group consisting of phenylbenzimidazole sulfonic acid and terephthalylidene dicamphor sulfonic acid. A ninth embodiment may provide the composition of the fifth embodiment, wherein the water-dispersible organic UV blocking agent is included in an amount of 1 wt % to 13 wt % relative to a total weight of the composition.

A tenth embodiment may provide the composition of the fifth embodiment, wherein the oil-dispersible organic UV blocking agent includes one or more selected from the group consisting of octocrylene, ethylhexyl salicylate, homosalate, bis-ethylhexyloxyphenol methoxyphenyl triazine, polysilicone-15, ethylhexyl triazone, butyl methoxydibenzoylmethane, drometrizole trisiloxane, and diethylamino hydroxybenzoyl hexyl benzoate.

An eleventh embodiment may provide the composition of the fifth embodiment, wherein the oil-dispersible organic UV blocking agent is included in an amount of 5 wt % to 35 wt % relative to the total weight of the composition.

A twelfth embodiment may provide the composition of the first embodiment, wherein a mixing weight ratio of the oil phase part to the water phase part is 1:1 to 10:1.

A thirteenth embodiment may provide the composition of the first embodiment, wherein the water phase part is thickened by further including a water phase gelling agent.

A fourteenth embodiment may provide the composition of the thirteenth embodiment, wherein the water phase gelling agent includes one or more selected from gums and mannan.

A fifteenth embodiment may provide the composition of the thirteenth embodiment, wherein the water phase gelling agent includes one or more selected from the group consisting of a carbomer, sodium polyacryloyldimethyl taurate, polyacrylate crosspolymer-6, xanthan gum, guar gum, locust bean gum, gellan gum, galactomannan, glucomannan, agar, carrageenan, a Sphingomonas ferment extract, acrylates/C10-30 alkyl acrylate crosspolymer, and an acrylates crosspolymer.

A sixteenth embodiment may provide the composition of the first embodiment, wherein the water phase part further includes a pH adjuster or neutralizer.

A seventeenth embodiment may provide the composition of the sixteenth embodiment, wherein the pH adjuster or neutralizer includes one or more selected from the group consisting of triethanolamine, aminomethyl propanol, ethanolamine, ammonium hydroxide, and tromethamine.

An eighteenth embodiment may provide the composition of the first embodiment, wherein the oil phase part further includes an elastomer.

A nineteenth embodiment may provide the composition of the eighteenth embodiment, wherein the elastomer is a silicone gel.

A twelfth embodiment may provide the composition of the nineteenth embodiment, wherein the silicone gel includes one or more selected from the group consisting of dimethicone/dimethicone crosspolymer, isododecane/dimethicone/bis-isobutyl PPG-20 crosspolymer, and dimethicone/vinyl dimethicone crosspolymer.

A twelve first embodiment may provide the composition of the eighteenth embodiment, wherein the elastomer is included in an amount of 5 wt % to 35 wt % relative to the total weight of the composition.

Hereinafter, the present disclosure will be described in more detail through Examples, and the like. These Examples are only for exemplifying the present disclosure, and it will be obvious to those skilled in the art that the scope of the present disclosure is not interpreted to be limited by these Examples.

### PREPARATION EXAMPLES

The preparation methods of the examples and comparative examples according to an embodiment of the present disclosure are as follows, and the components and contents of the examples and comparative examples are shown in the following [Table 1].

As an example of the present disclosure, the components of an oil phase part were sequentially put into a container and mixed and dissolved. After the components of a water phase part were mixed and dissolved in a separate container, the resulting mixture was introduced into the container including the components of the oil phase part and mixed using a homogenizer (Homo Mixer, Mark 2 2.5) to prepare compositions for blocking ultraviolet rays (Example 1 and Example 2).

As a comparative example of the present disclosure, after the components of an oil phase part were sequentially put into a container at room temperature and completely dissolved and dispersed, the components of a water phase part were mixed and dissolved in a separate container, and then the resulting mixture was introduced into the container including the components of the oil phase part and emulsified using a homogenizer (Homo Mixer, Mark2 2.5) to prepare compositions for blocking ultraviolet rays (Comparative Example 1 and Comparative Example 2).

TABLE 1

| | | | Composition | | | |
|---|---|---|---|---|---|---|
| | | | Example | Comparative | Example | Comparative |
| | | | 1 | Example 1 | 2 | Example 2 |
| | Function | Raw material name (INCI) | Content | Content | Content | Content |
| Oil | Solvent | Dibutyl adipate | TO 100 | TO 100 | TO 100 | TO 100 |
| phase | UV | Ethylhexyl salicylate | 4.5 | 4.5 | 4.5 | 4.5 |
| | blocking | | | | | |
| | agent | | | | | |
| | UV | Homosalate | 14 | 14 | 7 | 7 |
| | blocking | | | | | |
| | agent | | | | | |
| | UV | Bis-ethylhexyloxyphenol | — | — | 3 | 3 |
| | blocking | methoxyphenyl triazine | | | | |
| | agent | | | | | |
| | UV | Diethylamino hydroxybenzoic | — | — | 3 | 3 |
| | blocking | acid hexyl benzoate | | | | |
| | agent | | | | | |
| | Elastomer | Dimethicone crosspolymer | 15 | 15 | 10 | 10 |
| | Solvent | Butyloctyl salicylate | 10 | 10 | 10 | 10 |
| | Elastomer | Dimethicone/vinyl dimethicone | 0.5 | 0.5 | 0.5 | 0.5 |
| | | crosspolymer | | | | |
| | Film | Acrylates/polytrimethylsiloxymethacrylate | 5 | 5 | 5 | 5 |
| | former | copolymer | | | | |
| | Solvent | Dimethicone | 15 | 15 | 10 | 10 |
| | Preservative | Preservative | 0.2 | 0.2 | 0.2 | 0.2 |
| | Oil phase | Silica | 1 | 1 | 1.5 | 1.5 |
| | thickening | | | | | |
| | Emulsifier | PEG-10 Dimethicone | — | 3 | — | 3 |
| Water | Solvent | Ionized water | 20 | 20 | 20 | 20 |
| phase | Polyol | Polyol | 5 | 5 | 5 | 5 |
| | Preservative | Preservative | 1 | 1 | 1 | 1 |
| | UV | Phenylbenzimidazole sulfonic | 2.5 | 2.5 | 2.5 | 2.5 |
| | blocking | acid | | | | |
| | agent | | | | | |
| | Water | Xanthan gum | 0.05 | 0.05 | 0.05 | 0.05 |
| | phase | | | | | |
| | thickener | | | | | |
| | Water | Gellan gum | 0.05 | 0.05 | 0.05 | 0.05 |
| | phase | | | | | |
| | thickener | | | | | |
| | PH | Tromethamine | 2.2 | 2.2 | 2.2 | 2.2 |
| | adjuster | | | | | |
| | Water | Silica | 2 | 2 | 2 | 2 |
| | phase | | | | | |
| | thickening | | | | | |

### EXPERIMENTAL EXAMPLES

### 1. Comparative Experiment of UV Blocking Power

In accordance with the ‘UV Blocking Functionality Test Method’ in the ‘Standards and Test Methods of Functional Cosmetics’ notified by the Ministry of Food and Drug Safety, for the UV blocking index, after a transparent plastic tape (Transpore Tape; 3M, USA) was attached to a prepared cast, the cosmetic compositions of Examples 1 and 2 and Comparative Examples 1 and 2 were applied onto a sample at 2 mg/cm², and then the UV blocking effect was measured using a UV meter (SPF 290 Analyzer). The UV blocking index was summarized as the average value of three experimental values, and the results are shown in the 10 following [Table 2].

As can be seen from the values shown in the following [Table 2], it can be confirmed that Example 1 and Comparative Example 1, and Example 2 and Comparative Example 2, which include the same components and contents thereof except for the presence or absence of an emulsifier, each have similar SPF indexes.

TABLE 2

| | | Comparative | | Comparative |
|---|---|---|---|---|
| | Example 1 | Example 1 | Example 2 | Example 2 |
| Average SPF value | 23 | 25 | 29 | 30 |
| of UV test results | | | | |
| (n = 3) | | | | |

### 2. Comparative Experiment of Transparency

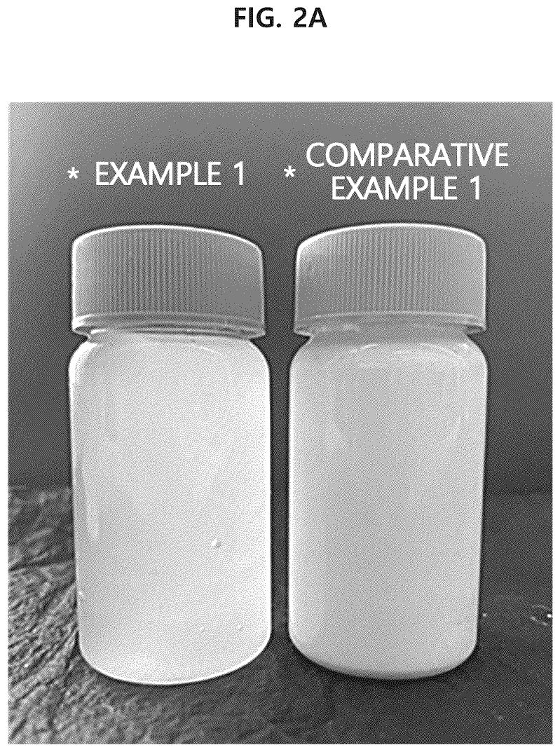

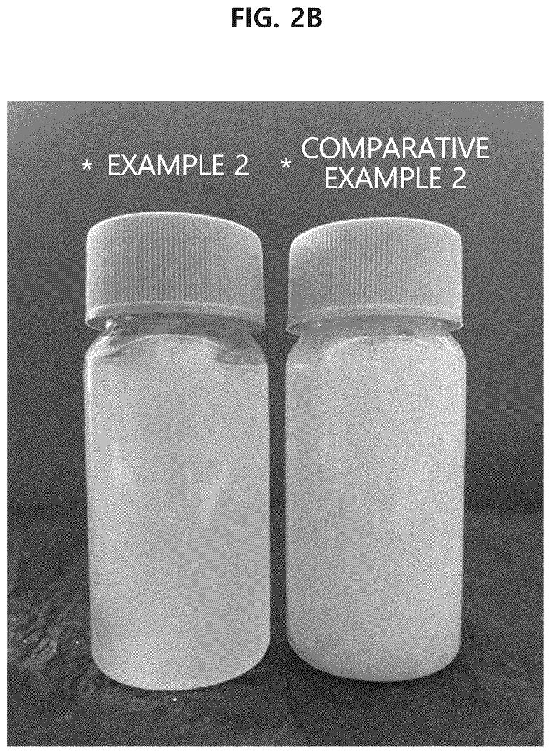

When Example 1 and Comparative Example 1 were placed in a transparent container, the transparency of the formulation appearance was compared with the naked eye, and as a result, it could be confirmed that Example 1 maintained transparency compared to the opaque Comparative Example 1 (FIGS. 2A and 2B).

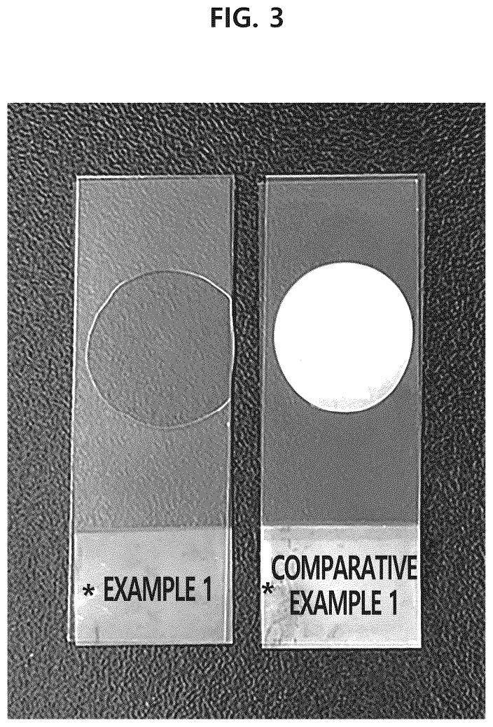

In addition, a predetermined amount of each of Example 1 and Comparative Example 1 was applied between slide glasses, and the transparencies of the formulations were compared when the slide glasses were overlapped and pressed together. It could be confirmed that Comparative Example 1 was applied opaquely, and thus, the color of the bottom surface could not be identified, whereas Example 1 was applied transparently, and thus, the color of the bottom surface under the slide glass was well illuminated (FIG. 3).

### 3. Comparative Experiment of Water Resistance

The formulation was applied to a slide glass at the same thickness using an applicator (100 μm), and dried at room temperature for 5 minutes, and then ionized water was dropped onto the applied surface using a dropper to confirm the shape of the water droplets spreading over the applied composition.

As a result of the experiment, it was confirmed that in the case of Comparative Example 1, the water droplets were dispersed on the formulation and the degree of spreading was high, whereas in Example 1, the water droplets were not dispersed well and the degree of spreading was low.

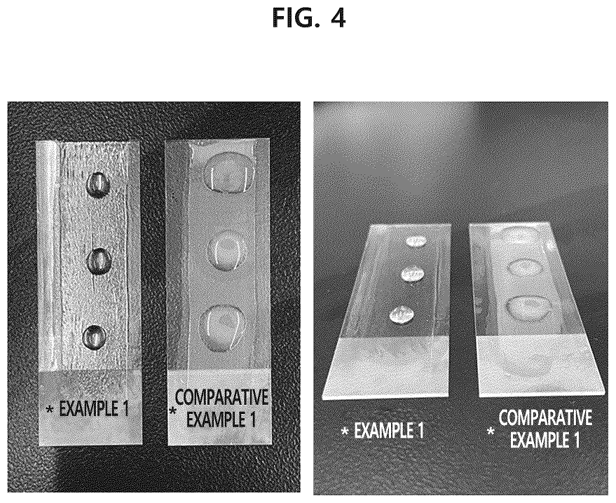

Therefore, it was confirmed that the water resistance of Example 1 was excellent compared to that of Comparative Example 1 (FIG. 4).

We claim:

1. A cosmetic composition comprising a water phase part and an oil phase part,
wherein the water phase part and the oil phase part are mixed, and
the cosmetic composition is a transparent formulation.

2. The composition of claim 1, wherein a viscosity of the composition is 2000 cps to 15000 cps (Brookfield viscometer) at 30° C.

3. The composition of claim 1, wherein the composition allows the water phase part and the oil phase part to be mixed to form a bicontinuous phase.

4. The composition of claim 1, wherein the water phase part comprises a water-dispersible organic UV blocking agent and a powder, and the oil phase part comprises an oil-dispersible organic UV blocking agent and a powder.

5. The composition of claim 4, wherein the powder is silica or cellulose.

6. The composition of claim 4, wherein a weight of the powder comprised in the water phase part is 0.5 wt % to 3.0 wt % relative to the entire composition, and a weight of the powder comprised in the oil phase part is 0.5 wt % to 3.0 wt % relative to the entire composition.

7. The composition of claim 4, wherein the water-dispersible organic UV blocking agent comprises one or more selected from the group consisting of phenylbenzimidazole sulfonic acid and terephthalylidene dicamphor sulfonic acid.

8. The composition of claim 4, wherein the water-dispersible organic UV blocking agent is comprised in an amount of 1 wt % to 13 wt % relative to a total weight of the composition.

9. The composition of claim 4, wherein the oil-dispersible organic UV blocking agent comprises one or more selected from the group consisting of octocrylene, ethylhexyl salicylate, homosalate, bis-ethylhexyloxyphenol methoxyphenyl triazine, polysilicone-15, ethylhexyl triazone, butyl methoxydibenzoylmethane, drometrizole trisiloxane, and diethylamino hydroxybenzoyl hexyl benzoate.

10. The composition of claim 4, wherein the oil-dispersible organic UV blocking agent is comprised in an amount of 5 wt % to 35 wt % relative to the total weight of the composition.

11. The composition of claim 1, wherein a mixing weight ratio of the oil phase part to the water phase part is 1:1 to 10:1.

12. The composition of claim 1, wherein the water phase part is thickened by further comprising a water phase gelling agent.

13. The composition of claim 12, wherein the water phase gelling agent comprises one or more selected from gums and mannan.

14. The composition of claim 12, wherein the water phase gelling agent comprises one or more selected from the group consisting of a carbomer, sodium polyacryloyldimethyl taurate, polyacrylate crosspolymer-6, xanthan gum, guar gum, locust bean gum, gellan gum, galactomannan, glucomannan, agar, carrageenan, a Sphingomonas ferment extract, acrylates/C10-30 alkyl acrylate crosspolymer, and an acrylates crosspolymer.

15. The composition of claim 1, wherein the water phase part further comprises a pH adjuster or neutralizer.

16. The composition of claim 15, wherein the pH adjuster or neutralizer comprises one or more selected from the group consisting of triethanolamine, aminomethyl propanol, ethanolamine, ammonium hydroxide, and tromethamine.

17. The composition of claim 1, wherein the oil phase part further comprises an elastomer; or
the oil phase part further comprises the elastomer, and the elastomer is comprised in an amount of 5 wt % to 35 wt % relative to the total weight of the composition.

18. The composition of claim 17, wherein the elastomer is a silicone gel.

19. The composition of claim 18, wherein the silicone gel comprises one or more selected from the group consisting of dimethicone/dimethicone crosspolymer, isododecane/dimethicone/bis-isobutyl PPG-20 crosspolymer, and dimethicone/vinyl dimethicone crosspolymer.

20. A method for blocking ultraviolet rays, the method comprising applying an effective amount of the cosmetic composition of claim 1 to the skin.
